(19) United States
(12) Patent Application Publication
(10) Pub. No.: US 2004/0126338 A1
(43) Pub. Date: Jul. 1, 2004

(54) Process of converting trans-lutein into cis-lutein and the uses thereof

(76) Inventors: Rod Ausich, Des Moines, IA (US); Jerry Newman, West Des Moines, IA (US); Amanda Ueltschy, Des Moines, IA (US); Holly Johnson, West Des Moines, IA (US); Fayad Z. Sheabar, West Des Moines, IA (US); Roger Dann, Des Moines, IA (US); Tatania Emmick, Milo, IA (US)

Correspondence Address: Davis, Brown, Koehn, Shors & Roberts, P.C., The Financial Center, Suite 2500, 666 Walnut Street, Des Moines, IA, 50309-3993, US

(21) Appl. No.: 10/331,801

(22) Filed: Dec. 30, 2002

(51) Int. Cl. A61K 35/78; A61K 7/42; A61K 7/06; C07C 403/00

(52) U.S. Cl. 424/59; 424/74; 424/747; 424/769; 585/351

(57) ABSTRACT

The invention consists of a process for producing new carotenoid compounds from lutein, which for the purposes of this description the new carotenoids are cis isomers of lutein, zeaxanthin, and lutein esters. The cis-isomer products are carotenoid compounds having UV absorption properties and are formed by preparing a solution of lutein and a liquid, refluxing the solution, cooling the solution to room temperature, and centrifuging the solution to remove trans-lutein crystals. The present invention particularly provides for a product, produced under mild conditions, of cis-isomers, which are preferred due to their increased UV absorption and lighter pigmentation, and thus creating an improved topically applied personal care product further having the same antioxidant characteristics as trans-isomers used before. The cis-isomer products may be used in topically applied personal care products or various food and beverage products and dietary supplements.

### BACKGROUND OF THE INVENTION

1. Field of the Invention

The invention relates generally to a process for the formation of cis-isomers of lutein and, more specifically, to a process for safely converting trans-isomers of lutein into cis-isomers of lutein for use in mixtures having high UV absorption.

2. Background of the Prior Art

Carotenoids are naturally-occurring yellow to red pigments of the terpenoid group that can be found in plants, algae, bacteria, and certain animals, such as birds and shellfish. The term “carotenoids” refers to a large class of molecules in which more than 600 naturally occurring carotenoids have been identified. Carotenoids include hydrocarbons (carotenes) and their oxygenated, alcoholic derivatives (xanthophylls). They include actinioerythrol, astaxanthin, canthaxanthin, capsanthin, capsorubin, β-8′-apo-carotenal (apo-carotenal), β-12′-apo-carotenal, α-carotene, β-carotene, “carotene” (a mixture of α- and β-carotenes), γ-carotenes, β-cyrptoxanthin, lutein, lycopene, violerythrin, zeaxanthin, and esters of hydroxyl- or carboxyl-containing members thereof. Many of the carotenoids occur in nature as cis- and trans-isomeric forms, while synthetic compounds are frequently racemic mixtures. The carotenes are commonly extracted from plant materials. For example, lutein extracted from marigold petals is widely used as an ingredient in poultry feed where it adds color to the skin and fat of the poultry and to the eggs produced by the poultry. Many of the carotenes are also made synthetically; much of the commercially available β-carotene has been made through synthesis.

Chemically, carotenoids are classified as “polyisoprenoid” molecules, meaning that they are synthesized by reactions that involve coupling together molecules of isoprene (also isopentenyl), an unsaturated 5-carbon molecule, therefore, most carotenoids and carotenoid precursors are known to contain multiples of 5 carbon atoms. The multiple unsaturated bonds allows them to absorb high-energy light waves in the blue and near-ultraviolet regions of the spectrum. The absorption of the high-energy light wavelengths of the blue and near-UV regions of the spectrum causes the carotenoids to reflect the wavelengths from other regions of the spectrum, thus carotenoids are usually yellow, orange, brown, or red in color. The carotenoid's color in solution may depend on the concentration, molecular structure, as well as other chemicals in the mixture. Carotenoids also have “conjugated” double bonds which means the double bonds alternate with single bonds, so that each carbon atom in a chain is double-bonded to one other carbon atom.

The level of conjugation in a compound depends on the carotenoids. For example, the carbon chains of β-carotene, lutein, and zeaxanthin are conjugated and each has alternating double and single bonds. However, structurally the beta-carotene and zeaxanthin have conjugation on the first bonds in both end rings, causing them to have the same chemical formulas but different properties, and lutein has different conjugation pattern altogether. Further, to illustrate the correlation between conjugation levels and protective activity, it should also be noted that a lycopene, the most highly conjugated of all carotenoids, is the best carotenoid oxygen quencher and has been identified as active in reducing the risk of prostate cancer (di Mascio et al. 1989).

Through evolutionary development carotenoids are designed to absorb the potentially harmful energy of blue and near-UV light and are therefore used as protective pigments by both plants and animals. Accordingly, since one of the functional goals of most plants is to absorb sunlight while still minimizing cellular and DNA damage caused by the harmful wavelengths, carotenoids are abundant throughout the plant kingdom. Ultraviolet radiation damage to plants is minimized by the carotenoids. Animals have also acquired ways to utilize carotenoids as photo-protective pigments, however because they cannot synthesize the carotenoids as plants do they must ingest the carotenoids from plant sources. One widely known example is β-carotene. Animals obtain the β-carotene from plant sources or from meat and once inside the body they convert it into other molecular forms such as vitamin A.

Carotenoids are valuable as pigments and as biologically active compounds therefore they are used in the pharmaceutical industry and as ingredients in nutritional supplements, most commonly to date because of their pro-vitamin A activity. They have been extensively studied as antioxidants for protection against cancer and other human and animal diseases. Among the dietary carotenoids, the focus has been on β-carotene. More recently, research has begun to elicit the broad role that other carotenoids play in human and animal health.

There are two main classes of carotenoids, carotenes that do not contain oxygen atoms, thus true hydrocarbons, and xanthophylls that do contain oxygen atoms. The xanthophylls in particular have been shown to possess strong antioxidant capabilities and may be useful in reducing the risk of disease. For example, the consumption of lutein and zeaxanthin, which are both reddish-orange pigments, are especially important because they are present in the retinas of mammals and most other animals. Zeaxanithin and lutein are the two primary pigments that give the macula (the circular region in the center of the retina) its characteristic yellow color and have been identified as leading to a 57 percent reduction in age-related macular degeneration (Seddon et al., 1994 J. Amer. Med. Assoc. 272(18): 1413-1420). Lutein, obtained mainly from marigolds, is commercially important as both an animal feed and food supplement. One wide use has been in feed for chickens in order to give the chicken's skin and egg yolks a deeper yellow color, which has been shown to appeal to consumers.

An aspect of carotenoid chemistry that is very important to the invention pertains to a field of chemistry involving “stereochemistry” and “stereoisomers.” A “chiral” atom is defined as a carbon atom having four of atoms or molecular groups attached to it. Lutein and zeaxanthin are stereoisomers of each other which can be seen at a carbon atom in each of the two end rings. Stereoisomers are carbon atoms that have the same constitution, but different disposition of groups in space. In other words the atoms are connected to each other in the same way, and differ only with respect to relative orientation in three-dimensional space. Therefore, the carbon atoms bonded to other atoms in a three-dimensional tetrahedral arrangement create stereoisomers; one of the stereoisomers rotates polarized light in a “right-handed” manner as the light passes through a liquid solution of the compound and the other arrangement causes the second stereoisomer to rotate polarized light in the opposite “left-handed” manner. The isomer that causes right-handed rotation is called the R stereoisomer and the isomer that causes left-handed rotation is referred to as the S stereoisomer. The S,S and R,R will always be enantiomers, meaning they must be mirror images of each other. Therefore, there are four possible stereoisomers of the carotenoids. One of these stereoisomers is the R-R isomer, in which both chiral carbon atoms have R configurations. Another stereoisomer is the S-S isomer, in which both chiral carbon atoms have the S configurations. The third and fourth isomers are the two “mixed” or “meso” (one R and one S chiral atom) isomers; the R-S isomer, and the S-R isomer. However, if the compound is symmetric (as in zeaxanthin), these two isomers are identical in every respect; if one draws the R-S isomer on paper, then one merely has to rotate the paper to generate the S-R isomer. In effect, “meso” isomers are formed by creating either of two stereoisomers (R-S or S-R). Therefore, because zeaxanthin is symmetric when it is synthesized the R-R isomer and the S-S isomer are present at a concentration of roughly 25% each, and the meso, or S-R combination, isomer is present at about 50% of the zeaxanthin, forming a racemic mixture.

Carotenoids are very specific in their cellular and enzymatic activities and are not interchangeable even though they have an identical chemical formula, as is seen with lutein and zeaxanthin. Therefore, lutein and zeaxanthin must be regarded as different and distinct carotenoids, each having its own isomers (i.e., stereoisomeric forms). Analytical methods such as chiral column chromatography (see, e.g., Bone, Landrum, et al, 1993) or circular dichroism analysis (see, e.g., Britton 1994) may be used to distinguish the stereoisomers.

Carotenoids have been of wide interest as a source of added color for food and drink products and many efforts have been made to attempt to use them as “natural” colorants for foods and beverages to increase their nutritional value. One area in which carotenoids are being used is in human multivitamin/multi-mineral products and dietary supplements, where they are typically added to tablet or capsule formulations. A problem with the use of carotenoids in food and personal care products, including supplement tablets, is the degradation of most carotenoids upon exposure to heat, oxygen, and light. Degradation will be accelerated at the elevated pressures used in tabletting. Typically a slight overage (10-30%) of the initial amount of the carotenoid is added to the tablet to meet the level desired allowing for losses during formation of the tablet. One method currently used commercially to increase the stability of the carotenoids is to microencapsulate the carotenoids in concentrations up to 10%. The inert coating materials increase the stability of the carotenoids during both the formation and shelf life of the tablet.

Prior art has attempted to address the heat and oxygen lability of the carotenoids by encapsulating the carotenoid source with a coating material mix in a fluidized bed, spray dryer, or similar microencapsulation process. While this technique may result in a certain level of activity of the active ingredient during tabletting, topical products do not use the technique, thus leading to the necessity of other methods for preserving the desired activity of lutein. The use of trans-carotenoids has been used in skin preparations, such as anti-oxidants, however, the antioxidant effectiveness of trans-carotenoids is difficult to utilize due to its intense coloring effect. Furthermore, the trans-carotenoids provide little direct protection from UV radiation.

Few attempts have been made in the prior art to study the different effects of trans-isomers as compared to cis-isomers in humans. In addition, previous studies entailed the use of harmful solvents and catalysts (e.g., I₂ crystals) and did not aim at forming cis-isomers or learning about their potential application. In a similar carotenoid, lycopene, the cis-lycopene was found to be more bioavailable than trans-lycopene and less likely to crystallize than the trans conformations. (Boileau, A. C., et al., 1999, J. Nutr. 129: 1176-1181). Further, it was noted that the majority of lycopene found in tomatoes and products was present as trans-lycopene while the lycopene in human and animal tissues contained significantly more cis-lycopene (Ibid.). Similarly, research using an HPLC equipped with a photodiodearry detector noted the presence of cis-lutein isomers in human plasma (Khachik, F., et al., 1992, J. of Chrom., 582: 153-166), and the trans-isomers of lutein, previously shown to be stable in crystalline forms. Upon reflux in ethanol, the cis-isomers were found to be easily reverted to the more thermodynamically stable trans-isomers.

As stated, the ultimate purpose of the above studies was not the formation of cis-isomers, and therefore conversion rates, yields, or the capability to produce large yields did not receive attention. Those that reported on the formation of cis-isomers of lutein created the isomers by heating the lutein in solvents such as benzene, hexane, chloroform and ethanol in the presence of crystalline iodine under the sun infrared or fluorescent light for several hours. These conditions entail the use of harmful solvents and iodine under special lighting conditions, such as infra red, sun, and fluorescent light for the preparation of the cis-lutein isomer. In addition, the potential application for cis-isomers was not foreseen or proposed in the prior art. Thus, a need exists for the preparation of cis-isomers using mild conditions to ensure the safe use of the products in various products such as those for personal care.

### SUMMARY OF THE INVENTION

The invention consists of methods for producing new carotenoid compounds from lutein, which for the purposes of this description the new carotenoids are cis-isomers of lutein and zeaxanthin. Suitable oils include corn oil, corn mint, spearmint oil, peppermint oil, bay oil, tea tree oil, and both red thyme oil and white thyme oil. Suitable solvents include hexane, ethanol, acetone, methylene chloride, tretrahydrofuran, and propylene glycol. The lutein may be from various sources but the lutein used in the following examples is dry cake lutein and centrate waste.

The new carotenoids are formed by preparing a solution of lutein in an oil or solvent. The solution is then refluxed, cooled, and centrifuged to remove the trans-isomers that have crystallized out of the solution during the cooling and the samples stored.

Products of this process may be used in topically applied personal care products or various food and beverage products and dietary supplements. The present invention particularly provides for a product, produced under mild conditions, of cis-isomers, which are preferred due to their increased UV absorption and lighter pigmentation, and thus creating an improved topically applied personal care product further having the same antioxidant characteristics as the previously used trans-isomers. Others have not attempted achieving these goals.

These and other objects of the present invention will become apparent to those skilled in the art upon reference to the following specification, drawings, and claims.

### BRIEF DESCRIPTION OF THE DRAWINGS

FIG. 1 is a chromatogram of trans-isomers.

FIG. 2 is a chromatogram of cis-isomers.

FIG. 3 is a graph showing the percentage of cis-lutein formed using the various solvents.

FIG. 4 is a diagram of the formation of dry cake lutein and centrate waste.

FIG. 5 is a graph of the trans-lutein spectrum.

FIG. 6 is a graph of the cis-lutein spectrum.

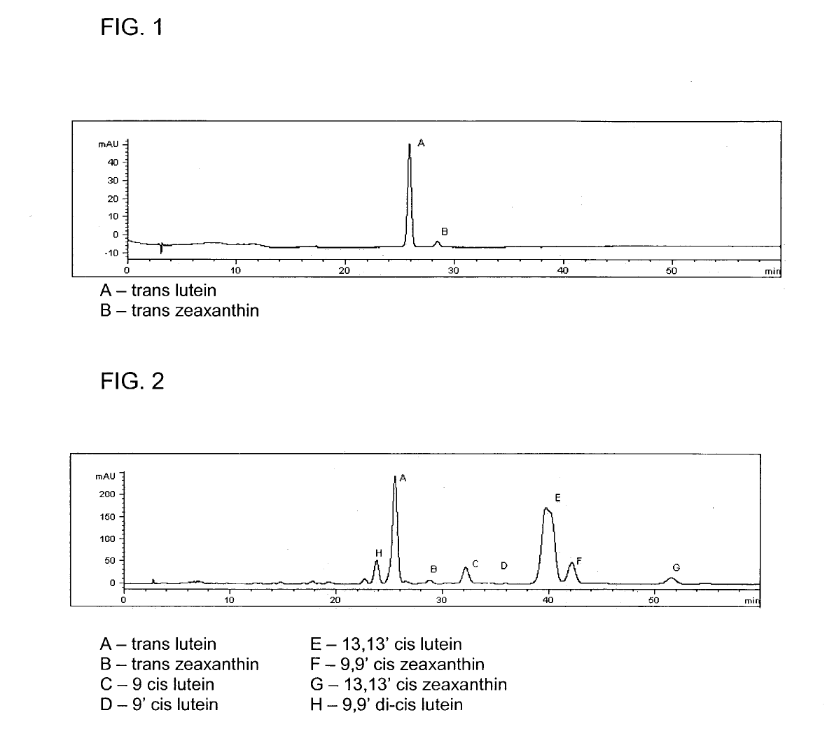

### DETAILED DESCRIPTION OF THE INVENTION

The cis-isomers of the present invention are cis-isomers of lutein, which have recently been discovered to have higher ultraviolet light (UV) absorption and a less pronounced orange color than their trans-isomer lutein counterparts (see FIGS. 7 and 8), while still displaying the antioxidant quality of trans-isomer lutein. The trans-isomers of carotenoids, although useful as antioxidants, have not been widely used in products for the skin because of their intensive color and lower UV absorption.

The lutein used in the method may be of any desired form that permits the lutein to go into solution. Preferably, the lutein will be freshly prepared or in a protected form so that it has a high activity. In particular the preferred embodiment of the lutein used for the following examples will be in a pure crystalline form or in the form of powder, referred to in the following disclosure as “dry cake” (Kemin Foods, L.C.)

The liquid used in the following method consists of mainly solvents or oils that are suitable to form a solution for the isomerization of lutein. The solvents are from a group comprising hexane, ethanol, acetone, methylene chloride, tetrahydrofuran (THF), and propylene glycol, and the oils are from a group comprising corn, corn mint, spearmint, peppermint, bay, and tea tree oil as well as both red thyme oil and white thyme oil. Red thyme and white thyme oils are produced from an herb that is from the plant family Lamiaceae (Labiateae). Red thyme oil is obtained by steam distillation of the leaves and flowers of the plant. The white thyme oil is then produced after a second distillation process has been carried out. Red thyme oil is brownish red in color and has a strong, spicy, warm smell, whereas white thyme oil is very pale yellow in color and smells sweeter and less pungent than red thyme oil.

The purpose of the following examples is to analyze the cis-isomerization that occurs when heat is supplied to a solution of lutein dissolved in a liquid. Therefore, the isomerization of lutein into cis-lutein by the incubation of lutein in liquids, such as corn oil, essential oils, or other solvents, was carried out and samples were collected at different time intervals in order to determine the concentration of the cis-isomers formed.

Using an analytical balance, the preparation of solution for the following examples for cis-isomerization includes placing weighed dry cake lutein into a flask and adding one of the above liquids to the flask to form the solution. The solution is first hand swirled and its solubility is observed. Following a baseline sample, which is taken at the 0 hour mark, the prepared lutein solution is placed on a heat source and refluxed for a period of time ranging between 0-120 hours.

The solvent solutions are refluxed in the following examples by placing the glass flask on a heating unit. A reflux column, which is cooled by non-recirculating cold water, is attached to the flask. The solution is then heated to the boiling point of the given liquid, except for propylene glycol, which boils at 187° C. The refluxing time varies slightly between solution samples. The refluxing times for each example are stated for each below. Samples are taken periodically to test the concentration of the cis-isomers, these are referred to as collected samples. Similarly, the oil solutions were heated in the following examples by placing the glass flask into a heated oil bath filled with mineral oil, which was stirred constantly, at 72° C.

Storing and centrifuging refers to the collected samples being placed into 12×75 mm glass culture tubes and wrapped with Parafilm® (Pechiney Plastic Packaging) and stored in a freezer until analysis. When prepped for analysis the collected samples are first warmed to room temperature and then the aliquot is centrifuged at 12,000 rpm for 5 minutes to remove trans-isomers crystals. The trans-isomer crystals are removed because the cis-isomers of lutein are known to be more soluble than the trans-isomers, which will allow them to remain in solution once formed. Next 1 ml of the centrifuged collected sample is placed into a High Performance Liquid Chromotography (HPLC) vial for analysis.

Analysis of the solutions formed from lutein and a liquid are analyzed using both normal phase HPLC and a spectophotometer. For the HPLC analysis, 1 ml of the collected sample of each solution is filtered through a 0.45 micron syringe filter into a HPLC sample vial. The sample is first dried using a nitrogen stream and then the remaining solids are dissolved in a 1 ml of a 65:35 hexane:ethylacetate mixture. The sample is sonicated for 2 minutes and injected onto the HPLC using a silica column (Agilent, Palo Alto, Calif.) with isocratic elution (65:35 hexane:ethylacetate) and detection at 446 nm. In isocratic elution compounds are eluted using constant mobile phase composition. As the sample solution flows through a column with the mobile phase, the components of the solution migrate according to the non-covalent interactions of the compound with the column, therefore it is the chemical interactions that determine the degree of migration and separation of components contained in the sample. In this way the percent area of the lutein cis-isomer peak is determined.

The solutions prepared in the experiments using hexane and ethanol as solvents with lutein are analyzed with a HPLC method using a 50 microliter sample which is run through a 250×3.0 mm Hypersil Silica column, the sample run having a flow rate of 0.250 ml per minute and a run time of 45 minutes. The dried sample is dissolved using a 65:35 hexane:ethylacetate mixture, as stated above. During the column run a backpressure of 43 bar is used with the analysis being done at 22° C. The visible lamp remains at 446 nm for detection.

Lutein cis-isomer %=(total lutein %×HPLC percent area for cis-isomers of lutein)

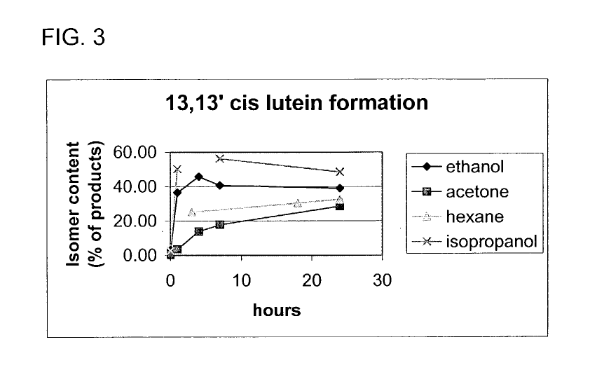

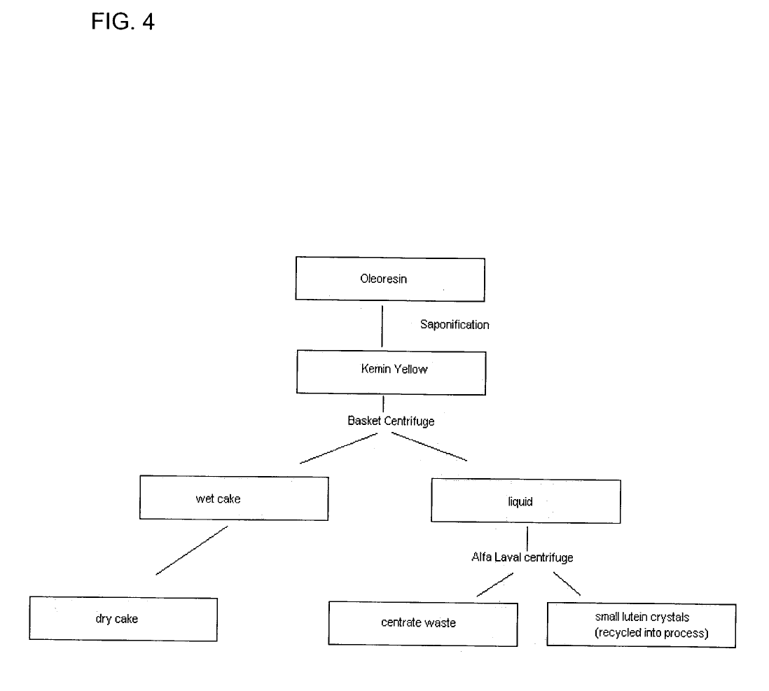

The HPLC results confirm that the isomerization from trans-isomers of lutein to cis-isomers of lutein has occurred. As seen in FIGS. 3 and 4, sample chromatograms show the trans-isomer peak to be present in all samples, while the various cis-isomer peaks (the 9,9′ cis-isomer peak and the 13,13′ cis-isomer peak) appears only after exposure to heat, which occurs during refluxing.

### EXAMPLE 1

### Formation of Cis-Isomers of Lutein and Zeaxanthin in Various Solvents

This experiment was conducted to analyze the effect of heat on a solution of lutein in a solvent selected from the group including hexane, ethanol, acetone, methylene chloride, Tetrahydrofuran (THF), and propylene glycol. It was then determined whether isomerization from trans-isomers of lutein to various cis-isomers of lutein occurs, and to what extent each isomer was formed. In this example 15 mg of lutein dry cake (15.7 mg in hexane and 15.6 mg in ethanol) and a 100 ml of solvent were added to each flask in order to prepare the solutions. The solutions were refluxed followed by the collection of 2 ml samples at the intervals of 1, 4, 8, and 24 hours.

The collected samples were centrifuged and analyzed. The amounts of each isomer are reported in weight:weight ratios and are shown in Table 1.

In general, the solvent producing the most cis-isomerization was hexane. Ethanol was the second highest producer. Propylene glycol showed a similar production of isomerization in the collected sample taken at four hours, but the cis-isomer content decreased after building up, indicating that the cis-isomer formed in this solution was not stable.

TABLE 1
Lutein isomers refluxed in a variety of solvents.
Hexane reflux (70° C.)

| Time (hr) | trans (%) | 9,9′ cis (%) | 13,13′ cis (%) | 9,9′ cis zea |
|---|---|---|---|---|
| 0 | 91.9 | 0 | 0 | 0 |
| 1 | 57.42 | 1.87 | 26.21 | 3.46 |
| 4 | 43.85 | 1.06 | 42.32 | 6.53 |
| 8 | 40.76 | 1.56 | 44.24 | 6.84 |
| 24 | 37.26 | 3.15 | 43.03 | 6.47 |

Ethanol reflux (81° C.)

| Time (hr) | trans (%) | 9,9′ cis (%) | 13,13′ cis (%) | 9,9′ cis zea |
|---|---|---|---|---|
| 0 | 91.9 | 0 | 0 | 0 |
| 1 | 57.42 | 1.87 | 26.21 | 3.46 |
| 4 | 43.85 | 1.06 | 42.32 | 6.53 |
| 8 | 40.76 | 1.56 | 44.24 | 6.84 |
| 24 | 37.26 | 3.15 | 43.03 | 6.47 |

Acetone reflux
(60° C.)

| Time | | | | | |
|---|---|---|---|---|---|
| point | trans lutein | trans zea | 9,9′ cis lut | 13,13′ cis lut | 9,9′ cis zea |
| 0 | 88.76 | 7.67 | 0.37 | 0.26 | 0.00 |
| 1 | 88.64 | 7.60 | 0.31 | 1.56 | 0.25 |
| 4 | 84.29 | 6.93 | 0.73 | 5.50 | 0.89 |
| 8 | 80.17 | 6.38 | 0.42 | 9.38 | 1.48 |
| 24 | 71.82 | 5.75 | 0.72 | 16.12 | 2.46 |

THF reflux (65° C.)

| Time | | | | | |
|---|---|---|---|---|---|
| point | trans lutein | trans zea | 9,9′ cis lut | 13,13′ cis lut | 9,9′ cis zea |
| 0 | 88.93 | 7.86 | 0.13 | 0.52 | 0.12 |
| 1 | 87.73 | 7.41 | 0.18 | 2.60 | 0.47 |
| 4 | 83.42 | 7.09 | 0.24 | 6.49 | 1.17 |
| 8 | 76.86 | 6.03 | 0.30 | 12.33 | 2.17 |
| 24 | 67.89 | 5.44 | 0.55 | 19.66 | 3.28 |

Dichloromethane reflux (41° C.)

| Time | | | | | |
|---|---|---|---|---|---|
| point | trans lutein | trans zea | 9,9′ cis lut | 13,13′ cis lut | 9,9′ cis zea |
| 0 | 90.42 | 7.58 | 0.19 | 0.21 | 0.00 |
| 1 | 90.37 | 7.50 | 0.14 | 0.38 | 0.00 |
| 4 | 89.59 | 7.38 | 0.14 | 1.06 | 0.21 |
| 8 | 88.79 | 7.39 | 0.17 | 1.71 | 0.32 |
| 24 | 86.63 | 7.05 | 0.19 | 3.84 | 0.67 |

Propylene glycol reflux (75° C.)

| Time | | | | | |
|---|---|---|---|---|---|
| point | trans lutein | trans zea | 9,9′ cis lut | 13,13′ cis lut | 9,9′ cis zea |
| 0 | 93.47 | 6.53 | 0.00 | 0.00 | 0.00 |
| 1 | 71.31 | 4.97 | 0.00 | 20.89 | 0.00 |
| 4 | 50.12 | 4.36 | 0.00 | 20.47 | 11.61 |
| 8 | 40.31 | 4.94 | 3.04 | 14.43 | 4.18 |
| 24 | 67.70 | 7.58 | 17.43 | 3.95 | 3.34 |

### EXAMPLE 2

### Formation of Cis-Isomers of Lutein and Zeaxanthin in Various Solvents

The purpose of this preparation is to analyze the effect of heat on a solution of lutein in four solvents, hexane, ethanol, isopropanol, and acetone, to determine whether isomerization from trans-isomers of lutein to various cis-isomers occurs, and what concentration of each isomer is formed. The solutions were prepared using 100 ml of the above stated solvents to dissolve varying weights of lutein dry cake. Initial solubility limits ere tested by adding a small amount of dry cake and swirling the flask briefly. If the sample appeared saturated, it was centrifuged to verify that a pellet of undissolved material was formed. If the solution was not saturated, additional dry cake was added until saturation was reached. The saturation data is listed in Table 2. Once the approximate solubility at room temperature was determined for each solvent, excess dry cake was added for the increased solubility that occurs through heating and isomerization, in order to maintain saturation during the entire experiment.

TABLE 2
Solvent solubility.

| | Room temp. sataturation | Total drycake | Reflux |
|---|---|---|---|
| | limit (%) | added (g) | temp (° C.) |
| Hexane | <1 | 4.0 | 63 |
| Ethanol | ˜0.3 | 1.4 | 78 |
| Isopropanol | ˜0.2 | 4.88 | 82 |
| Acetone | ˜0.45 | 2.38 | 56 |

The solutions were refluxed at the boiling point of each solvent for 24 hours with collected samples taken a 0, 1, 4, 7, and 24 hours. The collected samples were stored at −20° C. until analysis.

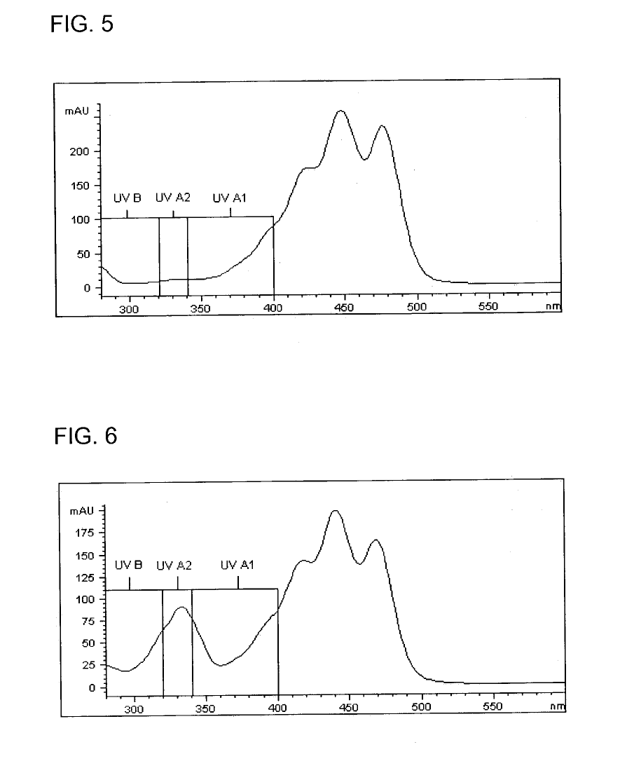

As shown in FIG. 5, different degrees of isomerization were found in the four solvents. The highest percentage of 13,13′ cis-lutein formation appears to have taken place in isopropanol but does not sustain the concentration over time, whereas, ethanol, which also has a significant 13,13′ cis-lutein formation, but not as high as the concentration found in isopropanol, is better able to sustain the concentration over a period of time. Neither hexane nor acetone were as effective in allowing isomerization to occur, but they did accumulate about 30% of the 13,13′ cis-lutein by the end of the refluxing time.

The data from the concentration analysis is shown in Table 3. The solubility of dry cake lutein in isopropanol increased dramatically when the solution was heated. However, once the experiment was complete and the solution cooled to room temperature, a thick sludge formed. Similar results were observed with the acetone solvent. Although the solubility of the acetone was not increased as dramatically when the solution was heated, the thick sludge was again observed when the acetone solution was cooled. The ethanol solution behaved quite differently once cooled. Rather than forming a thick sludge, upon cooling the ethanol solution formed crystals which were deposited on the side of the container and any remaining liquid settled to the bottom of the container. Analysis of the decanted liquid showed a mixture of cis and trans-isomers similar in concentration to the 24 hour time point, unlike the analysis of the crystals which were found to be predominantly trans-isomers.

In addition to analyzing the concentrations of the solutions, the total carotenoid content was calculated. Again, isopropanol was observed to have the highest content of the desired cis-isomer.

TABLE 3

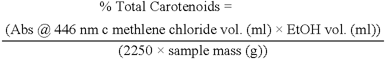

Isomer content, shown as % product (excluding degradation products).

| | % | % 13;13′ cis | % trans | % trans | % 9;9′ | % 13;13′ | % 9;9′ |
|---|---|---|---|---|---|---|---|
| | carotenoids | lug of total | lutein | zeaxanthin | cis lutein | cis lutein | cis zeaxanthin |
| Ethanol | | | | | | | |
| 0 | 0.0750 | 0.002 | 90.59 | 3.93 | 2.46 | 3.01 | 0.00 |
| 1 | 0.2783 | 0.102 | 44.19 | 1.12 | 6.42 | 36.57 | 7.09 |
| 4 | 0.3589 | 0.164 | 34.71 | 3.66 | 4.82 | 45.77 | 8.43 |
| 7 | 0.3981 | 0.162 | 34.39 | 3.73 | 4.29 | 40.72 | 8.17 |
| 24 | 0.4907 | 0.192 | 38.84 | 3.76 | 9.11 | 39.08 | 7.12 |
| Acetone | | | | | | | |
| 0 | 0.4045 | 0.000 | 93.85 | 6.15 | 0 | 0 | 0 |
| 1 | 0.5321 | 0.019 | 92.47 | 3 | 0 | 3.55 | 0 |
| 4 | 0.6631 | 0.091 | 79.81 | 3.63 | 0 | 13.77 | 2.79 |
| 7 | 0.7496 | 0.133 | 71.03 | 3.44 | 4.28 | 17.76 | 3.5 |
| 24 | 1.1411 | 0.327 | 60.45 | 3.5 | 0 | 28.66 | 5.3 |
| Hexane | | | | | | | |
| 0 | 0.0700 | 0.000 | | | | | |
| 1 | 0.3480 | 0.000 | | | | | |
| 3 | 0.5111 | 0.128 | 67.89 | 3.93 | 0.00 | 25.06 | 3.12 |
| 18 | 1.0484 | 0.319 | 63.03 | 2.98 | 3.13 | 30.43 | 0.43 |
| 24 | 1.7261 | 0.564 | 61.81 | 3.14 | 2.02 | 32.69 | 0.35 |
| Ispropanol | | | | | | | |
| 0 | 0.1373 | 0.004 | 91.48 | 5.93 | 0.00 | 2.59 | 0.00 |
| 1 | 0.7082 | 0.356 | 27.02 | 3.08 | 6.51 | 50.22 | 9.93 |
| 4 | 1.1470 | 0.000 | 0.00 | | | | |
| 7 | 1.1564 | 0.651 | 24.78 | 3.02 | 2.08 | 56.29 | 10.62 |
| 24 | 1.8017 | 0.873 | 28.16 | 2.63 | 8.82 | 48.48 | 9.11 |

### EXAMPLE 3

### Formation of Cis-Isomers of Lutein and Zeaxanthin in Essential Oils at 72° C. (2% Lutein)

The purpose of this preparation is to analyze the effect of heat on a solution of lutein in the above stated oils and determine whether isomerization from trans-lutein to various cis-isomers occurs, and the concentration of each isomer that is formed. The oils used in this example were corn oil, which was used as a control, corn mint oil, spearmint oil, peppermint oil, tea tree oil, and bay oil. Two different experiments were conducted, the first preparation had a solution using 3 mg of lutein dry cake in each of the above-identified oils and the second preparation had a solution using 4 mg of the lutein dry cake in each of the oils. The solutions were made by adding 20 ml of oil to the weighed lutein. The sample heated in an oil bath. A baseline 2 ml sample was taken and additional 2 ml samples taken at 1, 4, 8, 24, and 48 hours. The first trial, utilizing 3 mg of lutein, had an additional 2 ml sample taken at the 120 hour interval and the second trial, having 4 mg of lutein, had an additional 2 ml sample taken at the 72 hour interval.

In both trials it was observed that lutein was less soluble in corn oil than in the other oils. Further, in the trial using 4 mg of lutein, the corn oil was completely saturated throughout the experiment. When 3 mg of lutein was used there was not any remaining solid lutein in the solutions of other oils after a brief swirling. However, when 4 mg of lutein was used some solid remained in the solution until after a period of heating, after which all lutein was dissolved. In both trials the baseline samples were orange with the additional samples similar in color. In the corn mint samples, the color decreased slightly over time. Since the carotenoid content was not tracked, it is not known how much of the total lutein content decreased due to breakdown. Also of interest, no crystals formed when the samples were cooled which may indicate that trans-lutein was not crashing out of solution. Due to the low lutein concentration obtained from the first trial using 3 mg of lutein, focus will be on results from the second trial, using 4 mg of lutein.

The HPLC results confirmed that isomerization from trans-lutein to cis-lutein occurred in all oils. The lutein trans-isomer peak is present in all samples, while the various lutein cis-isomer peaks appeared only after a period of exposure to heat. Corn oil, which was included in the example as a ‘non-reactive’ control, showed the highest degree of isomerization, with a 42.22% concentration of 9,9′cis-lutein at 24 hours. However, due to the initial low solubility of lutein in corn oil, the other oils should be due to a higher efficiency. The remaining samples had comparable amounts of cis-isomers generated, and ranged from concentrations having 18-35% cis-isomers at 24 hours. Interestingly, the 13,13′ cis-isomer did not appear to be highly stable for extended periods. This was indicated by the concentration increase of the 13,13′cis-isomer up to the 24 hour sample followed by a decline in the concentration level. The HPLC chromatograms also showed an increase in degradation compounds in the samples taken from the later time points.

The 13,13′cis-lutein was the predominantly produced isomer in all oils, except bay oil. In the bay oil sample, a significant amount of 13,13′cis-isomer was initially generated. This was followed by a decrease in the amount of 13,13′ cis-isomer and an increase in the concentration of 9,9′cis-isomer. After 48 hours the concentrations of 13,13′cis-isomers and 9,9′cis-isomers reached an equilibrium. However, at 71 hours the 9,9′ isomer content became higher than the 13,13′ isomer content. Thus, when the lutein 9,9′cis-isomer is the desired product bay oil presents better yield.

The amounts of each isomer are presented as proportions in Table 4 below.

TABLE 4
Lutein isomers refluxed in a variety of oils.

| | trans | | | 13,13′ cis | |
|---|---|---|---|---|---|
| Sample oils | lutein | tran zea | 9,9′ cis lut | lut | 9,9′ cis zea |
| Corn | | | | | |
| 0 | 95.31 | 4.03 | 0.00 | 2.12 | 0.00 |
| 1 | 70.05 | 2.90 | 0.78 | 22.04 | 4.23 |
| 4 | 50.31 | 2.76 | 0.85 | 38.81 | 7.27 |
| 7 | 58.30 | 3.23 | 0.87 | 31.79 | 5.81 |
| 24 | 44.99 | 2.70 | 0.00 | 42.22 | 7.43 |
| 49 | 59.05 | 3.74 | 7.38 | 25.41 | 4.12 |
| 71 | 65.09 | 4.23 | 1.05 | 22.16 | 3.91 |
| Corn mint | | | | | |
| 0 | 88.64 | 9.40 | 0.00 | 1.96 | 0.00 |
| 1 | 75.10 | 6.65 | 0.00 | 15.23 | 3.02 |
| 4 | 63.29 | 7.35 | 0.00 | 24.85 | 4.51 |
| 7 | 62.96 | 7.51 | 0.00 | 25.09 | 4.44 |
| 24 | 63.67 | 7.27 | 0.00 | 23.29 | 4.24 |
| 49 | 63.73 | 4.01 | 2.72 | 19.79 | 3.36 |
| 71 | 78.05 | 3.97 | 2.26 | 15.71 | 0.00 |
| Spearmint | | | | | |
| 0 | 88.49 | 9.90 | 0.00 | 1.62 | 0.00 |
| 1 | 75.35 | 7.60 | 0.00 | 14.13 | 2.91 |
| 4 | 62.96 | 7.36 | 1.28 | 23.86 | 4.48 |
| 7 | 62.24 | 7.76 | 1.45 | 24.08 | 4.39 |
| 24 | 63.05 | 6.56 | 2.89 | 21.86 | 3.89 |
| 49 | 61.78 | 6.53 | 6.65 | 21.54 | 3.50 |
| 71 | 63.39 | 6.72 | 4.92 | 21.40 | 3.56 |
| Peppermint | | | | | |
| 0 | 87.37 | 10.17 | 0.00 | 2.47 | 0.00 |
| 1 | 72.63 | 6.61 | 0.85 | 16.59 | 3.31 |
| 4 | 60.87 | 7.79 | 1.51 | 25.18 | 4.56 |
| 7 | 60.61 | 7.78 | 1.67 | 25.39 | 4.45 |
| 24 | 63.09 | 6.67 | 2.77 | 22.14 | 3.79 |
| 49 | 65.14 | 5.12 | 3.26 | 22.62 | 3.86 |
| 71 | 64.55 | 5.41 | 4.10 | 22.25 | 3.69 |
| Tea Tree | | | | | |
| 0 | 87.21 | 9.95 | 0.00 | 2.36 | 0.48 |
| 1 | 73.52 | 6.76 | 0.00 | 16.49 | 3.22 |
| 4 | 59.22 | 8.19 | 1.34 | 26.26 | 4.69 |
| 7 | 59.07 | 8.12 | 1.57 | 26.52 | 4.59 |
| 24 | 64.88 | 5.87 | 2.72 | 22.71 | 3.83 |
| 49 | 63.87 | 5.44 | 4.85 | 22.17 | 3.66 |
| 71 | 62.53 | 5.91 | 6.34 | 21.65 | 3.57 |
| Bay | | | | | |
| 0 | 84.57 | 11.64 | 0.00 | 3.79 | 0.00 |
| 1 | 68.75 | 6.15 | 2.87 | 18.55 | 3.67 |
| 4 | 56.31 | 8.10 | 6.45 | 24.14 | 4.45 |
| 7 | 53.98 | 8.06 | 8.58 | 24.26 | 4.39 |
| 24 | 58.40 | 6.34 | 13.17 | 18.73 | 3.36 |
| 49 | 55.58 | 7.26 | 17.03 | 17.09 | 3.04 |
| 71 | 54.00 | 7.37 | 19.03 | 16.59 | 3.00 |

### EXAMPLE 4

### Formation of Cis Lutein and Zeaxanthin Isomers in Various Thyme Oils at 72° C. (10% Lutein)

The purpose of this preparation is to analyze the effect of heat on a solution of lutein in the above-stated thyme oils and to determine whether isomerization from trans-lutein to various cis-isomers occurs, and the concentration of each isomer formed. The oils used were corn oil, which was the control, and both red thyme and white thyme oils obtained from Spectrum Oils and the Lebermuth Company. This experiment utilized a higher lutein concentration than the one used in the precious examples.

In this preparation a solution was made using 2 g of lutein dry cake and 20 ml of oil. The solution was heated in an oil bath. Samples (2 ml) were taken at 0, 1, 4, 6.5, 24, and 48 hours. As before, samples were analyzed qualitatively using normal phase HPLC and spectophotometer. However, quantitative analysis was also performed, by taking 1 ml of the HEAT (hexane, ethanol, acetone and toluene) solution previously prepared, diluting it to 25 ml in ethanol, and determining the absorbance on an UV-Vis absorption spectrophotometer. This type of analysis is often useful for quantitative measurements. All of the thyme oil samples required a 1:20 dilution of the ethanol solution before reading on the UV-Vis.

It was observed that the dry cake was the least soluble in corn oil and, as in the essential oils, some lutein solid remained in the other oil solutions even after swirling by hand. However, following a period of heating of less than an hour all the lutein was dissolved in each solution. Again, no crystals formed when the samples were cooled, indicating that lutein trans-isomers were not crashing out of solution. The baseline corn oil samples were orange, with all additional samples similar in color. The thyme oil samples, both the red and white oils, were dark red in color throughout the experiment.

HPLC results confirmed that isomerization from trans-isomers to cis-isomers occurred in all the oils. The trans-isomer peak was present in all samples, while the various cis-isomer peaks appeared only after a period of exposure to heat. Corn oil, which was included as a “non-reactive” control, showed the highest degree of isomerization, with a 38.45% concentration of 9,9′ cis-isomer content at 48 hours. The remaining samples were all comparable to each other in terms of the amount of cis-isomers generated and ranged between 17-18% at the 24 hour sample. Because the concentration of 13,13′cis-isomer is fairly constant from 4-48 hours the 13,13′ cis-isomer appears to be stable. All samples, except for the Spectrum White thyme oil, generated significant amounts of 9:9′ cis-isomers. In the samples taken at 48 hours the concentration of 9:9′ cis-isomers was still increasing, while the 13:13′ cis-isomer formation had reached a plateau.

The general cis-isomer conversion characteristics of these oils are very similar to conversion characteristics seen in the essential oil (mints, bay, tea tree) of rapid 13:13′ cis-isomer formation which reaches a plateau at 4 hours. The amounts of each isomer are presented as proportions and shown below in Table 5.

TABLE 5
Lutein isomers heated in a variety of oils.

| | | | | | |
|---|---|---|---|---|---|
| Time | | | | | |
| point | trans lut | trans zea | 9,9′ cis lut | 13,13′ cis lut | 9,9′ cis zea |

Corn Oil

| | | | | | |
|---|---|---|---|---|---|
| 0 | 92.60 | 5.59 | 0.00 | 1.80 | 0.00 |
| 1 | 80.92 | 4.53 | 1.42 | 11.05 | 2.07 |
| 4 | 62.30 | 3.30 | 0.00 | 29.35 | 5.05 |
| 6.5 | 56.67 | 3.19 | 1.99 | 32.76 | 5.40 |
| 24 | 57.87 | 2.78 | 2.86 | 31.86 | 4.65 |
| 48 | 49.40 | 2.20 | 3.66 | 38.45 | 6.28 |

Spectrum Red Thyme Oil

| | | | | | |
|---|---|---|---|---|---|
| 0 | 92.86 | 6.61 | 0.00 | 0.53 | 0.00 |
| 1 | 72.19 | 10.85 | 0.97 | 13.23 | 2.77 |
| 4 | 66.02 | 5.07 | 8.09 | 17.95 | 2.87 |
| 6.5 | 65.21 | 5.29 | 8.60 | 18.19 | 2.72 |
| 24 | 67.16 | 5.46 | 6.48 | 18.22 | 2.70 |
| 48 | 62.32 | 5.80 | 11.22 | 17.84 | 2.81 |

Spectrum White Thyme Oil

| | | | | | |
|---|---|---|---|---|---|
| 0 | 90.58 | 8.75 | 0.00 | 0.67 | 0.00 |
| 1 | 73.18 | 10.99 | 1.53 | 11.84 | 2.34 |
| 4 | 68.72 | 9.76 | 0.00 | 18.31 | 3.20 |
| 6.5 | 69.47 | 5.30 | 5.97 | 16.69 | 2.57 |
| 24 | 66.52 | 5.28 | 7.07 | 18.40 | 2.72 |
| 48 | 65.26 | 5.29 | 7.61 | 18.96 | 2.89 |

Lebermuth Red Thyme Oil

| | | | | | |
|---|---|---|---|---|---|
| 0 | 88.50 | 10.53 | 0.00 | 0.97 | 0.00 |
| 1 | 73.80 | 11.68 | 1.15 | 11.17 | 2.21 |
| 4 | 68.18 | 5.31 | 6.84 | 17.05 | 2.62 |
| 6.5 | 74.18 | 8.11 | 3.27 | 10.98 | 3.46 |
| 24 | 65.25 | 4.79 | 9.58 | 17.77 | 2.61 |
| 48 | 61.54 | 5.27 | 13.05 | 17.52 | 2.62 |

Lebermuth White Thyme Oil

| | | | | | |
|---|---|---|---|---|---|
| 0 | 91.47 | 7.38 | 0.00 | 1.15 | 0.00 |
| 1 | 72.32 | 10.40 | 1.78 | 12.82 | 2.56 |
| 4 | 65.59 | 5.04 | 9.44 | 17.41 | 2.52 |
| 6.5 | 65.14 | 5.07 | 7.95 | 18.72 | 2.93 |
| 24 | 65.80 | 5.27 | 8.49 | 17.81 | 2.63 |
| 48 | 62.77 | 5.03 | 11.55 | 17.92 | 2.74 |

The total carotenoid content is also shown in Table 6 below. The corn oil control was saturated throughout the experiment. As expected, the carotenoid content increased slightly during the first hour of heating, and remained fairly constant. Similarly, the thyme oil samples showed increased carotenoid concentration at 1 hour when compared to the baseline sample. Since all of the dry cake was dissolved, the total carotenoid content decreased over time as degradation occurred. The predicted concentration of 10% was not reached, however, a concentration of 7.18% was observed.

Overall, lutein in thyme oil was stable for up to about 6.5 hours. It was observed that the carotenoid content had significantly decreased by 24 or 48 hours, however, this was still higher than initially determined. Based on Table 6, the cis-isomer content at 24 hours is 25-30%, indicating that of the approximately 5% carotenoid content, only about 3.5% is trans-lutein.

TABLE 6
Carotenoid concentration over time.

| | Time (hr) | A446 | Dilution Factor | % Carotenoids |
|---|---|---|---|---|
| Corn Oil | | | | |
| | 0 | 0.12585 | 1 | 0.086 |
| | 1 | 0.20416 | 1 | 0.139 |
| | 4 | 0.22630 | 1 | 0.154 |
| | 6.5 | 0.27428 | 1 | 0.187 |
| | 24 | 0.21491 | 1 | 0.146 |
| | 48 | 0.30039 | 1 | 0.205 |
| Spectrum Red Thyme Oil | | | | |
| | 0 | 0.41085 | 20 | 4.630 |
| | 1 | 0.55792 | 20 | 6.287 |
| | 4 | 0.59102 | 20 | 6.660 |
| | 6.5 | 0.58661 | 20 | 6.610 |
| | 24 | 0.56800 | 20 | 6.401 |
| | 48 | 0.56036 | 20 | 6.315 |
| Spectrum White Thyme Oil | | | | |
| | 0 | 0.42557 | 20 | 4.818 |
| | 1 | 0.59660 | 20 | 6.754 |
| | 4 | 0.55198 | 20 | 6.249 |
| | 6.5 | 0.54828 | 20 | 6.207 |
| | 24 | 0.50549 | 20 | 5.723 |
| | 48 | 0.50032 | 20 | 5.664 |
| Lebermuth Red Thyme Oil | | | | |
| | 0 | 0.47841 | 20 | 5.492 |
| | 1 | 0.58816 | 20 | 6.752 |
| | 4 | 0.57154 | 20 | 6.561 |
| | 6.5 | 0.62544 | 20 | 7.180 |
| | 24 | 0.51864 | 20 | 5.954 |
| | 48 | 0.46179 | 20 | 5.301 |
| Lebermuth White Thyme Oil | | | | |
| | 0 | 0.38594 | 20 | 4.420 |
| | 1 | 0.61384 | 20 | 7.030 |
| | 4 | 0.54418 | 20 | 6.233 |
| | 6.5 | 0.64057 | 20 | 7.337 |
| | 24 | 0.51360 | 20 | 5.882 |
| | 48 | 0.43436 | 20 | 4.975 |

### EXAMPLE 5

### Formation of Cis Lutein and Zeaxanthin Isomers Using Various Media and Heat

The purpose of this preparation was to examine the conversion of trans-lutein to cis-lutein in solutions of corn oil, water, and Spectrum brand white thyme oil when the lutein is heated. The experiment also looked at a 25% w/v (w/v% is defined as weight solute (g)/volume solution (ml)×100) solution of lutein in various medias. The example includes a sample of dry cake heated in the absence of liquid in order to observe the effect of heat by itself.

Samples containing 25% solutions were prepared using 2.5 g dry cake lutein and 10 ml of liquid. The liquid used in solution was from the group consisting of corn oil, diH₂O, or Spectrum brand white thyme oil. The “dry” sample contained 2.5 g dry cake lutein and was preformed without the addition of any liquid. The flasks placed in an oil bath. Samples were removed at 0, 1, 4, and 24 hours for analysis by UV-Vis and HPLC.

The sample aliquots were weighed into 25 ml volumetric flasks and 0.5-4 ml of HEAT solution was transferred to another 25 ml flask and diluted with ethanol. A portion of this solution was read using the UV-Vis (see Table 7). A 1 ml portion of each HEAT solution was also dried using nitrogen and reconstituted using a 65:35 hexane:ethyl acetate mixture prior to analysis by HPLC to show the relative amounts of each carotenoid.

The data in Table 7 shows that isomerization does not readily occur in lutein samples heated in the absence of liquid. The data further shows that the cis-isomers of lutein is either not formed, or not soluble in water, when lutein is heated in a solution of only water. Data again shows that lutein has both a good solubility and conversion to cis-isomers when heated in white thyme oil.

TABLE 7
Conversion of lutein to cis-isomers in various media.

| | | | | | | | | | | % 9;9′ |
|---|---|---|---|---|---|---|---|---|---|---|
| | | ml HEAT | | | % | % trans | % trans | % 9;9′ | % 13;13′ | cis |
| | g sample | into EtOH | dilution | A446 | carotenoids | lutein | zeaxanthin | cis lutein | cis lutein | zeaxanthin |
| Corn Oil | | | | | | | | | | |
| 0 | 0.234 | 5 | 1 | 0.21934 | 0.04595 | 85.61 | 6.91 | 2.74 | 2.85 | 0.75 |
| 1 | 0.2288 | 4 | 1 | 0.62678 | 0.16786 | 81.65 | 7.19 | 2.22 | 5.05 | 1.01 |
| 4 | 0.2381 | 4 | 1 | 0.87905 | 0.22622 | 72.39 | 6.83 | 2.82 | 14.93 | 2.58 |
| 24 | 0.95 | 4 | 20 | 0.21448 | 0.27668 | 70.56 | 7.72 | 0.00 | 1.71 | 0.00 |
| Water | | | | | | | | | | |
| 0 | 0.4742 | 3 | 1 | 0.32481 | 0.05596 | 79.28 | 6.45 | 6.03 | 6.58 | 1.15 |
| 1 | 0.2767 | 4 | 3 | 0.53795 | 0.35738 | 87.49 | 10.34 | 0.67 | 1.06 | 0.24 |
| 4 | 0.2522 | 2 | 1 | 0.14022 | 0.06814 | 100.00 | 0.00 | 0.00 | 0.00 | 0.00 |
| Spectrum White | | | | | | | | | | |
| Thyme Oil | | | | | | | | | | |
| 0 | 0.3098 | 0.5 | 5 | 0.43581 | 3.44791 | 100.00 | 0.00 | 0.00 | 0.00 | 0.00 |
| 1 | 0.0896 | 0.5 | 2 | 0.60452 | 6.61458 | 82.52 | 6.61 | 0.00 | 8.55 | 1.69 |
| 4 | 0.0711 | 0.5 | 1 | 0.77059 | 5.31280 | 69.81 | 4.76 | 3.17 | 17.88 | 3.23 |
| 24 | 0.2032 | 1 | 20 | 0.36851 | 8.88987 | 64.90 | 5.12 | 3.03 | 21.93 | 3.87 |
| Dry Heated | | | | | | | | | | |
| 0 | 0.0167 | 0.5 | 5 | 0.42111 | 61.80433 | 100.00 | 0.00 | 0.00 | 0.00 | 0.00 |
| 1 | 0.0082 | 0.5 | 1 | 0.9296 | 55.57150 | 82.23 | 7.94 | 4.42 | 4.12 | 0.72 |
| 4 | 0.0051 | 0.5 | 1 | 0.60373 | 58.02864 | 84.26 | 7.14 | 4.47 | 4.12 | 0.00 |

### EXAMPLE 7

### Sample Analysis of Carotenoid Content from Centrate Waste

The purpose of this example was to analyze a sample of centrate waste for carotenoid content and recover the carotenoids using a variety of solvents, the solvents being from the group comprising hexane, ethanol, isopropanol, acetone, and methylene chloride. Centrate waste is part of the product that is removed and discarded during the production of dry cake lutein. FIG. 6 is a diagram showing the formation of dry cake and centrate waste. The selected solvents were used to form solution samples using three different ratios with the centrate waste, the ratios being 3:1. 1:1, and 1:3.

The samples were prepared by weighing a small amount of the centrate waste into a volumetric flask and diluting the sample with HEAT. The 1-4 ml sample was transferred to another volumetric flask and diluted with ethanol and analyzed by reading at A446.

A series of solutions were set up using a solvent from the above-identified group to prepare 4 ml of each sample of centrate waste at the ratios of 3:1, 1:1, and 1:3. It was observed that the expected layers were not formed in the ethanol, acetone, and isopropyl samples. The hexane and methylene chloride did exhibit expected distinct layers and an emulsion formed which was removed by adding a small amount of NaCl. After centrifugation the hexane layer was aspirated and a portion was diluted with ethanol and the absorbance was determined by a Uv-Vis spectrophotometer. The supernatant was aspirated and a small amount of sodium sulfate was added to remove any remaining moisture. The dried methylene chloride was diluted using ethanol and analyzed using the UV-Vis spectrophotometer.

As seen in Table 8, some of the solvents were miscible with the water based centrate, therefore they were not useful to remove the carotenoids from. The remaining samples, when mixed with the useful solvents, showed a color gradient over the series of dilutions; the 3:1 solvent:centrate was much lighter colored than the 1:3 solvent:centrate. This information can help in optimization of the extraction process.

As seen in Table 9, the carotenoid content extracted from the samples increases as the proportion of centrate increases. In the 1:1, methylene chloride :centrate solution, the percent of carotenoids is similar to that of the original centrate sample. This indicated that almost 100% extraction is occurring. While hexane is able to extract carotenoids from the centrate, the efficiency is much lower than that of methylene cholride.

TABLE 8
Solvent: centrate mixtures.

| | Layers | Layers | Layers after | |
|---|---|---|---|---|
| Sample | (Y/N) | after vortex | centrifuging | Comments |
| Hexane 3:1 | Y | Y | Y | Emulsion layer |
| Hexane 1:1 | Y | Y | Y | Emulsion layer |
| Hexane 1:3 | Y | Y | Y | Emulsion layer |
| EtOH 3:1 | N | N | N | Small Pellet |
| EtOH 1:1 | Y | N | N | Small Pellet |
| EtOH 1:3 | Y | N | N | Small Pellet |
| Isopropanol 3:1 | N | N | N | Cloudy |
| Isopropanol 1:1 | N | N | N | Very cloudy |
| Isopropanol 1:3 | N | N | N | Very cloudy |
| Acetone 3:1 | N | N | N | Small Pellet |
| Acetone 1:1 | N | N | N | Small Pellet |
| Acetone 1:3 | Y | N | N | Small Pellet |
| MeCl₂ 3:1 | Y | N | Y | Emulsion layer |
| MeCl₂ 1:1 | Y | N | Y | Emulsion layer |
| MeCl₂ 1:3 | Y | N | Y | Emulsion layer |

TABLE 9
Sample analysis.

| Sample | Weight | ml HEAT into EtOH | A446 | % Carotenoids |
|---|---|---|---|---|
| 52401CentA | 0.5376 | 2 | 0.1965 | 0.0448 |
| 52401CentB | 0.5253 | 2 | 0.1826 | 0.0426 |

| Sample | Volume (mcl) | Weight | ml EtOH Solution | A446 | % Carotenoids |
|---|---|---|---|---|---|
| MeCl₂ 3:1 | 100 | 0.1132 | 4 | 0.5984 | 0.00829 |
| MeCl₂ 1:1 | 30 | 0.0340 | 4 | 0.6721 | 0.03105 |
| MeCl₂ 1:3 | 10 | 0.0113 | 4 | 0.7053 | 0.09776 |
| Hexane 3:1 | 100 | 0.0663 | 4 | 0.1789 | 0.00423 |
| Hexane 1:1 | 50 | 0.0332 | 4 | 0.1204 | 0.00570 |
| Hexane 1:3 | 60 | 0.0398 | 4 | 0.3092 | 0.01219 |

### EXAMPLE 8

### Sample Analysis of Carotenoid Content from Centrate Waste

The purpose of this example is to analyze a sample of centrate waste for carotenoid content and recover the carotenoids using a variety of solvents, the solvents being from the group comprising hexane, methylene chloride, and ethyl acetate. Centrate samples were diluted with solvents at 3:1. 1:1, and 1:3 ratios. The centrate sample used in this example was extracted using the procedure discussed above in Example 7.

Again a 4 ml sample was made using the centrate and solvent. All three samples had distinct layers and any emulsion formed was removed by adding a small amount of NaCl. After centrifugation, the hexane and ethyl acetate layers were aspirated. The methylene chloride layer was withdrawn from below the aqueous layer and a small amount of sodium sulfate was added to each sample to remove any residual moisture. The dried solvents were stored at −20° C. until analysis. The 1:3 ratios for methylene chloride and ethyl acetate extractions were repeated using 8 ml volume samples to increase the solvent recovery for analysis.

Table 10 shows the recorded data from the extraction process. As seen before, the extracted solvents demonstrated a color gradient over the series of dilutions; the 3:1 solvent:centrate was much lighter colored than the 1:3 solvent:centrate.

TABLE 10
Solvent: centrate mixtures.

| | | Layers | | |
|---|---|---|---|---|
| | Layers | after | Layers after | |
| Sample | (Y/N) | Vortex | Centrifuging | Comments |
| Hexane 3:1 | Y | Y | Y | Emulsion layer |
| Hexane 1:1 | Y | Y | Y | Emulsion layer |
| Hexane 1:3 | Y | Y | Y | Emulsion layer |
| MeCl₂ 3:1 | Y | N | Y | Emulsion layer |
| MeCl₂ 1:1 | Y | N | Y | Emulsion layer |
| MeCl₂ 1:3 | Y | N | Y | Emulsion layer |
| Ethyl acetate 3:1 | Y | Y | Y | Aqueous layer |
| | | | | still orange |
| Ethyl acetate 1:1 | Y | N | N | Entire sample is |
| | | | | light orange, no |
| | | | | layers |
| Ethyl acetate 1:3 | Y | N | N | ˜0.5 ml aqueous |
| | | | | layer on top |

### EXAMPLE 9

### Re-Exploring the Extraction of Centrate Waste using Hexane, Methylene Chloride, and Ethyl Acetate

The purpose of this example was to re-analyze an extraction of centrate waste for carotenoid content and recover the carotenoids using a variety of solvents, the solvents being from the group comprising hexane, methylene chloride, and ethyl acetate. The selected solvents used with the samples were examined using three different ratios, the ratios being 3:1. 1:1, and 1:3 solvent:centrate. The centrate sample used in this example was extracted using the procedure discussed above in Example 7.

Solutions were prepared by diluting the weighed centrate samples with solvents from the group comprising hexane, methylene chloride, and ethyl acetate into 3:1, 1:1, and 1:3 ratios of solvent:centrate. As observed above, two distinct layers were formed after addition of the solvent to the aqueous centrate. The samples were vortexed and approximately 50 mg sodium sulfate was added to remove the emulsion. The samples were centrifuged briefly. The solvent layer with the extracted carotenoids was transferred to a clean tube and diluted with ethanol. The absorbance of this solution was determined using a UV-Vis spectrophotometer at 446 nm and the percentage of carotenoids was calculated. Both the UV-Vis and percentage of carotenoids are shown in Table 11. The data shows that methylene cholride and ethyl acetate are much better at extracting carotenoids from the centrate waste than hexane. An advantage of using ethyl acetate is that it is less toxic than methylene cholride.

TABLE 11
Centrate extraction using solvents.

| | | | | % |
|---|---|---|---|---|
| Sample | Dilution Factor | Further Dilution | A446 | Carotenoids |
| Hex 3:1 | 3.5 | | 0.37740 | 0.00052 |
| Hex 1:1 | 3.5 | | 0.59743 | 0.00082 |
| Hex 1:3 | 7.3 | | 1.06060 | 0.00302 |
| MetCl₂ 3:1 | 9.3 | 6.0 | 0.48208 | 0.01059 |
| MetCl₂ 1:1 | 13.5 | 8.5 | 0.73750 | 0.03319 |
| MetCl₂ 1:3 | 26.0 | 51.0 | 0.23885 | 0.12420 |
| EthAcet 3:1 | 6.0 | 6.0 | 0.37252 | 0.00526 |
| EthAcet 1:1 | 9.3 | 51.0 | 0.26530 | 0.04952 |
| EthAcet 1:3 | 26.0 | 51.0 | 0.29893 | 0.15544 |

### EXAMPLE 10

### Sample Analysis of Carotenoid Content from Centrate Waste

The purpose of this example was to examine the extraction of centrate waste for carotenoid content and recover the carotenoids using a variety of essential oils, the oils being selected from the group comprising corn oil, spearmint, peppermint, corn mint, bay oil, tea tree oil, red thyme oil, and white thyme oil. The solutions (4 ml) were made with the selected oil at three different ratios, 3:1. 1:1, and 1:3 (oil:centrate). The centrate sample used in this example was extracted using the procedure discussed in Example 7.

The samples were vortexed and centrifuged. The oil layer found in the spearmint oil, peppermint oil, bay oil, red thyme oil, and white thyme oil was diluted with ethanol and absorbance determined on a UV spectomometer at 446 nm. The corn oil and tea tree oils had too large of an emulsion for analysis. The samples with corn oil and bay oil were not soluble and, therefore, were not read on the UV-Vis spectomometer. The results are shown below in Table 12.

In general, the thyme oils appeared to extract the most lutein. Only red thyme oil showed an increase in the concentration of lutein above the concentration observed in the centrate.

TABLE 12
Centrate extraction using oils.

| | | | After | Initial Dilution | Further | | % |
|---|---|---|---|---|---|---|---|
| Sample Oils | 2 Layers | After Vortex | Centrifuge | in EtOH | Dilution | A446 | Carotenoids |
| Corn 3:1 | yes | partial separation | large emulsion | not soluble | — | — | |
| Corn 1:1 | yes | partial separation | large emulsion | not soluble | — | — | |
| Corn 1:3 | yes | partial separation | large emulsion | not soluble | — | — | |
| Spearmint 3:1 | yes | partial separation | 2 layers | 9 | 7.7 | 0.18099 | 0.00979 |
| Spearmint 1:1 | yes | partial separation | 2 layers | 9 | 21.0 | 0.20672 | 0.03064 |
| Spearmint 1:3 | yes | partial separation | 2 layers | 9 | 21.0 | 0.28410 | 0.04211 |
| Peppermint 3:1 | yes | only 1 layer | 2 layers | 9 | 3.0 | 0.09468 | 0.00201 |
| Peppermint 1:1 | yes | partial separation | 2 layers | 9 | 5.0 | 0.24871 | 0.00878 |
| Peppermint 1:3 | yes | partial separation | 2 layers | 9 | 11.0 | 0.48524 | 0.03768 |
| Bay 3:1 | yes | partial separation | 2 layers | not soluble | — | — | |
| Bay 1:1 | yes | only 1 layer | 2 layers | not soluble | — | — | |
| Bay 1:3 | yes | only 1 layer | 2 layers | not soluble | — | — | |
| Tea tree 3:1 | yes | partial separation | 2 layers | 9 | 7.7 | 0.20745 | 0.01123 |
| Tea tree 1:1 | yes | partial separation | large emulsion | 9 | 11.0 | 0.73019 | 0.05670 |
| Tea tree 1:3 | yes | partial separation | large emulsion | 9 | 21.0 | 0.22553 | 0.03343 |
| Red Thyme 3:1 | yes | partial separation | 2 layers | 9 | 11.0 | 0.19811 | 0.01538 |
| Red Thyme 1:1 | yes | partial separation | 2 layers | 9 | 21.0 | 0.41544 | 0.06158 |
| Red Thyme 1:3 | yes | partial separation | 2 layers | 9 | 41.0 | 0.49219 | 0.14245 |
| White Thyme 3:1 | yes | only 1 layer | 2 layers | 9 | 7.7 | 0.21990 | 0.01190 |
| White Thyme 1:1 | yes | only 1 layer | 2 layers | 9 | 14.3 | 0.44873 | 0.04540 |
| White Thyme 1:3 | yes | only 1 layer | 2 layers | 9 | 21.0 | 0.39560 | 0.05864 |

### EXAMPLE 11

### Formation of Cis-Isomers using Isopropanol

The purpose of this preparation was to analyze the effect of isopropanol in solution with lutein, and analyze the effect of adding water to the lutein/isopropanol solution. In this example a solution was prepared by adding 2.51 g of dry cake lutein (lot 082405-08) to 50 ml isopropanol to a flask. The prepared solution was swirled and refluxed at 81° C. for 8 hours.

Samples were taken at 0, 1, 4, 6, and 8 hours to follow the isomerization. At 8 hours the sample was transferred to a 150 ml Erlenmeyer flask and placed in a freezer to cool. When partially cooled, aliquots were taken for precipitation and samples were diluted to 2:1, 1:1, and 1:2 ratios of water:isopropanol. An additional sample was prepared which contained only isopropanol solution. The samples were vortexed and centrifuged. Supernatant and pellets were collected and stored in a 1.5 ml microfuge tube until analysis.

As shown from the data in Table 13, isomerization occurred. Table 13 shows a higher cis-isomer concentration in the supernatants than in the pellets, thus confirming that the solubility of cis-isomers is higher than trans-isomers. It was also observed that crashing lutein out of solution by adding water to the isopropanol solution was not effective, as proven by the following two observations. First the water:isopropanol mixtures formed suspensions rather than distinct pellets or supernatants, as would have been expected if the lutein had crashed out of solutions Second, the cis-isomer concentration of the “supernatant” collected from the water:isopropaol mixture was not as high as the cis-isomer concentration of the “supernatant” collected from only the isopropanol, which did form a pellet with a transparent, dark red supernatant.

TABLE 13
Cis-isomer formation over time in supernatants, pellets, and precipitated aliquots.

| Sample | di-cis lut | trans lut | trans zea | 9 cis lut | 9′ cis lut | 13;13′ cis lut | 9;9′ cis zea | 13;13′ cis zea | total cis |
|---|---|---|---|---|---|---|---|---|---|
| 0 hr supernatant | 2.83 | 90.76 | 6.41 | 0.00 | 0.00 | 0.00 | 0.00 | 0.00 | 0.00 |
| 1 hr supernatant | 1.72 | 35.30 | 1.52 | 0.50 | 0.70 | 48.43 | 8.12 | 3.71 | 61.46 |
| 4 hr supernatant | 4.18 | 24.01 | 0.75 | 0.42 | 1.31 | 55.77 | 10.08 | 3.49 | 71.07 |
| 6 hr supernatant | 3.62 | 38.62 | 1.49 | 1.68 | 0.00 | 43.89 | 7.84 | 2.86 | 56.27 |
| 8 hr supernatant | 4.47 | 26.46 | 0.85 | 2.37 | 0.00 | 53.71 | 9.13 | 3.00 | 68.21 |
| 0 hr pellet | 0.00 | 94.58 | 5.42 | 0.00 | 0.00 | 0.00 | 0.00 | 0.00 | 0.00 |
| 4 hr pellet | 1.20 | 79.88 | 4.62 | 0.00 | 0.00 | 12.15 | 2.14 | 0.00 | 14.29 |
| 8 hr pellet | 1.27 | 75.91 | 4.16 | 0.75 | 0.00 | 14.68 | 2.10 | 1.13 | 18.66 |
| 2:1 H₂O:IPA sup | 2.30 | 65.36 | 3.13 | 1.28 | 0.00 | 22.91 | 3.46 | 1.56 | 29.21 |
| 1:1 H₂O:IPA sup | 2.17 | 64.99 | 3.15 | 1.40 | 0.00 | 23.59 | 3.23 | 1.45 | 29.67 |
| 1:2 H₂O:IPA sup | 2.43 | 64.05 | 3.09 | 1.51 | 0.00 | 23.95 | 3.15 | 1.82 | 30.43 |
| cold IPA sup a | 3.50 | 46.33 | 1.41 | 2.23 | 0.00 | 38.54 | 5.34 | 2.63 | 48.74 |
| cold IPA sup b | 4.09 | 37.94 | 1.03 | 2.36 | 0.00 | 45.57 | 6.18 | 2.83 | 56.94 |
| 2:1 H₂O:IPA pellet | 1.94 | 66.02 | 3.34 | 1.56 | 0.00 | 22.54 | 3.28 | 1.32 | 28.70 |
| 1:1 H₂O:IPA pellet | 2.22 | 66.08 | 3.10 | 1.01 | 0.00 | 23.20 | 2.89 | 1.47 | 28.57 |
| 1:2 H₂O:IPA pellet | 2.00 | 65.79 | 3.34 | 1.24 | 0.00 | 22.88 | 3.18 | 1.57 | 28.87 |
| cold IPA pellet | 1.96 | 66.33 | 3.89 | 1.15 | 0.00 | 21.60 | 3.68 | 1.38 | 27.81 |

The purpose of the invention described above is to modify an existing product, lutein, into its cis-lutein isomers using a new process to create a product having less color, higher UV absorbance at a very harmful range, and the same antioxidant characteristics as the previously known trans-lutein isomers, thus allowing the cis-isomers to be used, for example, in personal care products and a wide variety of other products. The above examples demonstrated the ability to generate significant amounts of cis-isomers using a relatively simple conversion process that utilizes either dry cake lutein or centrate waste. The results suggest ethyl acetate and white thyme provide the best extractions. White thyme further provides less UV interference than the red thyme, which had more interference due to the oil color present already present.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

We claim:

1. A process for safely forming cis-isomer lutein compounds having UV absorption properties, comprising the steps of:
a) preparing a solution of lutein and a liquid;
b) heating while refluxing the solution;
c) cooling the solution to room temperature; and
d) centrifuging the solution to remove trans-lutein crystals.

2. The process as defined in claim 1, further comprising the step of removing the solvent in the solution by drying.

3. The process as defined in claim 1, wherein the liquid is a solvent selected from the group comprising hexane, isopropanol, ethanol, acetone, methylene chloride, and ethyl acetate.

4. The process as defined in claim 1, wherein the liquid is an oil.

5. The process as defined in claim 4, wherein the oil is selected from the group comprising corn oil, corn mint, spearmint, peppermint, tea tree, and bay oil.

6. The process as defined in claim 1, wherein the oil is further selected from the group comprising red thyme and white thyme oils.

7. The process as defined in claim 1, wherein the lutein source is dry cake lutein.

8. The process as defined in claim 1, wherein the lutein source is centrate waste.

9. The process as defined in claim 1, wherein the cis-isomer carotenoid compounds further having antioxidant properties.

10. A process for forming cis-isomer lutein compounds having UV absorption properties, comprising the steps of:
a) preparing a solution by adding lutein to an essential oil, and
b) heating and incubating the solution.

11. A composition of a cis-isomer lutein compounds, comprising a product formed by the process of claim 1.

12. A personal care composition, comprising a cis-isomer lutein compound product of claim 11 added to a personal care product.

13. An antioxidant carotenoids composition having reduced coloring efficacy, comprising a cis-lutein product formed by the process of claim 1.
